(19) United States
(12) Patent Application Publication
(10) Pub. No.: US 2017/0253901 A1
(43) Pub. Date: Sep. 7, 2017

(54) METHOD FOR PRODUCING A COMPOUND OF THE STEROL BIOSYNTHESIS PATHWAY IN A EUKARYOTIC ORGANISM

(71) Applicants: INSTITUT NATIONAL DE LA RECHERCHE AGRONOMIQUE, PARIS CEDEX 07 (FR); CENTRE INTERNATIONAL D'ETUDES SUPERIEURES EN SCIENCES AGRONOMIQUES, MONTPELLIER (FR); UNIVERSITE DE MONTPELLIER, MONTPELLIER (FR); CENTRE NATIONAL DE LA RECHERCHE SCIENTIFIQUE, PARIS (FR)

(72) Inventors: PIERRE BERTHOMIEU, MONTPELLIER (FR); ORIANE MITH, MONTPELLIER (FR); LAURENCE MARQUES-BASTIDE, MONTPELLIER (FR); VÉRONIQUE PERRIER, VENDRES (FR); ERIC DUBREUCQ, MONTPELLIER (FR)

(21) Appl. No.: 15/512,534

(22) PCT Filed: Sep. 16, 2015
(86) PCT No.: PCT/FR2015/052485

§ 371 Date: Mar. 17, 2017

(30) Foreign Application Priority Data
Sep. 17, 2014 (FR) 1458763

(51) Int. Cl. C12P 33/00; C12N 1/18; C07K 14/415

(52) U.S. Cl. CPC C12P 33/00; C07K 14/415; C12N 1/18

(57) ABSTRACT

The invention concerns a method for producing a compound of interest of the sterol biosynthesis pathway in a eukaryotic organism or a derivative of a compound of the sterol biosynthesis pathway in said eukaryotic organism, in particular a sterol. This method comprises a step for the in vitro culturing of cells of said eukaryotic organism modified to express or overexpress a defensin, in a culture medium suitable for the development of said cells and containing at least one element chosen from the transition metals, lead and selenium, at a concentration greater than or equal to the concentration of said element that is necessary for inducing the overproduction of said compound of interest by said cells.

The present invention is part of the field of controlling the expression of metabolic pathways in eukaryotic organisms. More particularly, it relates to a method for producing a compound of interest synthesized by a eukaryotic organism in the sterol biosynthesis pathway, or derived from a compound synthesized by a eukaryotic organism in this sterol biosynthesis pathway. The present invention also relates to recombinant eukaryotic cells particularly suited to carrying out such a method, and also to the use of recombinant yeast cells for the production of a compound of interest of the sterol biosynthesis pathway or derived from a compound of the sterol biosynthesis pathway.

Engineering of the metabolism of organisms constitutes a growing field in biotechnology. The control of metabolic pathways makes it possible especially to produce or overproduce compounds of interest, to alter cellular characteristics, to increase the performance of processes using these organisms, etc.

In particular, numerous studies have been carried out to increase the yield of the sterol biosynthesis pathway in various types of organisms, especially in yeasts, by various strategies such as overexpression of genes involved in this biosynthesis pathway and/or optimization of fermentation conditions. Such studies are in particular described in the publication by Wriessnegger et al., 2013. A major aim of such studies is to increase the production yield, by the yeasts, of a particular sterol, ergosterol, which has proven to be an economically important product, especially since it constitutes a precursor to vitamin D2 and cortisone.

The processes proposed by the prior art with the aim of increasing the production yield of sterols by yeasts, or by other organisms, nonetheless have drawbacks, especially a complexity of implementation, a high cost, and insufficient purity of the target compound produced, and/or an insufficient level of production of this target compound.

The present invention aims to overcome the drawbacks of the methods for activating the sterol biosynthesis pathway proposed by the prior art, especially the drawbacks set out above, by proposing an alternative method for activating this biosynthesis pathway in a eukaryotic organism which is simple and inexpensive to implement and which makes it possible to overproduce, with a high yield, a compound of interest produced by this organism in the sterol biosynthesis pathway or a compound of interest derived from a compound produced by this eukaryotic organism in the sterol biosynthesis pathway.

In the present description the phrase “compound of interest derived from a compound produced by a eukaryotic organism in the sterol biosynthesis pathway” is intended to mean a compound obtained by biotransformation of said compound produced in the sterol biosynthesis pathway by an enzyme or a plurality of enzymes expressed by said eukaryotic organism, whether natively or following modification(s) of said eukaryotic organism.

The origin of the invention is the fact that it was discovered by the present inventors, within the context of a project with an entirely different goal, that quite surprisingly the culture of eukaryotic cells, in particular yeasts, genetically modified to express or overexpress a defensin, especially a defensin of plant origin, in the presence of an excess of certain elements in the culture medium leads to a significant modification of the cell metabolism, resulting especially in the overproduction of lipids and more particularly of sterols. The present inventors thus discovered that such cell culture conditions induce the expression of the vast majority of the genes involved in the sterol biosynthesis pathway. In yeast, it was especially established by the present inventors that the cells expressing a plant defensin encoded by a gene of the Plant Defensin type 1 family, subjected to an excess of zinc in the cell culture medium, for example to a concentration of zinc in this culture medium of approximately 20 mM, accumulate a large amount of sterols. The unsaponifiable fraction of the hexane-extractable lipids, that is to say of the neutral lipids, is thus composed to more than 80% of sterols. Such an accumulation is especially associated with an activation of the genes ERG3, ERG25, ERG27, ERG8 and MVD1 and also the genes ERG1, ERG2, ERG9, ERG10, ERG13, ERG20, ERG24 and ERG26 of yeast, involved in the ergosterol biosynthesis pathway, and also the gene UPC2, which is an activator of this pathway. The sterol biosynthesis pathway in yeast is in particular partially described in the publications by Burg et al., 2011, and Shobayashi et al., 2005.

Thus, according to the present invention, a method is proposed for producing a compound of interest of the sterol biosynthesis pathway in a eukaryotic organism, that is to say a compound synthesized by this eukaryotic organism in the sterol biosynthesis pathway, or a compound derived from a compound of the sterol biosynthesis pathway in said eukaryotic organism, by activating the sterol biosynthesis pathway in said eukaryotic organism. This method comprises a step of in vitro culture of cells of this organism expressing a defensin, in particular modified to express or overexpress a defensin, in a culture medium adapted to their growth and containing at least one element chosen from the transition metals, lead and selenium. This element is present in excess in the culture medium, that is to say, according to the present invention, at a concentration greater than or equal to the concentration of said element which is necessary to induce the overproduction of said compound of interest, in particular of said compound of the sterol biosynthesis pathway, by said cells. Overproduction is intended to mean the production, by said cells, of an amount of said compound of interest, in particular of said compound of the sterol biosynthesis pathway, at least 2 times greater than the amount produced by said cells under identical culture conditions, when the culture medium contains said element at the minimum concentration necessary for the optimal growth of said cells.

For each element considered, chosen from the transition metals, lead and selenium, the minimum concentration in the culture medium necessary for the optimal growth of the cells depends not only on the element itself, but also on the eukaryotic organism in question, and also on the general composition of the culture medium and more generally the culture conditions. It is within the skills of those skilled in the art, for each given situation, to determine this minimal concentration on the basis of their general knowledge. To this end, they may especially employ one of the methods developed in the prior art for the optimization of the composition of culture media.

For example, for each element in question, the minimal concentration to be provided in the culture medium in order to meet metabolic requirements and ensure optimal growth of the cells may be determined by the method referred to as “pulse and shift” which is one of the most effective methods proposed in the prior art. According to this method, the minimal concentration of a given element is determined during a culture carried out in a chemostat. The initial concentration value tested is generally obtained by elemental analysis of the biomass being studied. This method was initially described by Kuhn et al., 1979. By way of example, the use of this method to optimize a culture medium of Staphylococcus galinarum was described in the publication by Medaglia and Panke, 2010.

By way of illustration, the minimum zinc concentration of a commercial synthetic medium such as Yeast Nitrogen Base (DIFCO) necessary for the optimal growth of the yeast Saccharomyces cerevisiae (for 10 g/l of glucose, i.e. a final biomass of 0.1 to 5 g/l depending on the culture conditions) is 2.5 μM.

For some elements among the transition metals, lead and selenium, the minimal concentration necessary for the optimal growth of the cells may be 0, that is to say that the culture medium must be devoid thereof to ensure optimal growth, or even sometimes any growth at all, of the cells. This is the case for example for the transition metal cadmium.

It is also part of the skills of those skilled in the art to determine, for each element in question, chosen from the transition metals, lead and selenium, the concentration of said element which is necessary to make it possible to trigger the overproduction of the compound of interest, in particular of said compound of the sterol biosynthesis pathway, by the eukaryotic cells expressing defensin, that is to say, according to the invention, to enable the amount of the compound of interest, in particular of said compound of the sterol biosynthesis pathway, produced by the cells to be at least 2 times greater than the amount of this compound produced by the same cells under identical culture conditions except for the fact that the culture medium contains the element in question at the minimal concentration necessary for the optimal growth of the cells. Culture conditions are intended to mean the amount of biomass being cultured, the composition of the culture medium, the duration and the temperature of the culturing step, etc.

This concentration of said element making it possible to trigger in eukaryotic cells the metabolic response corresponding to the overexpression of the sterol biosynthesis pathway in the presence of defensin may for example be determined by a method for analyzing the effect of different concentrations of said element: either on the expression of the genes involved in the sterol biosynthesis pathway by comparison of the transcriptome of cells cultured on the one hand on medium containing said element at the minimum concentration and on the other hand on medium enriched in said element; or on the increase of the concentration of sterols or metabolites associated stoichiometrically with the sterol concentration, by comparison of the composition of cells cultured on the one hand on medium containing said element at the minimum concentration and on the other hand on medium enriched in said element.

The determination may be carried out by the “pulse and shift” method described above. It may also be carried out more simply, for example by carrying out parallel batch cultures of the strain producing the defensin in media containing increasing concentrations of said element, starting from the minimum concentration necessary for the optimal growth of the cells.

The culture medium used according to the invention also comprises the substances necessary for cell growth and for sterol biosynthesis. This medium may be liquid or solid. The culture conditions, and especially the culture temperature, are dependent on the particular eukaryotic organism in question, and defining them is part of the skills of those skilled in the art.

The transition metal present in the culture medium may especially be zinc, cadmium, nickel, cobalt or copper.

It may otherwise be chosen from scandium, titanium, vanadium, chromium, manganese, iron, yttrium, zirconium, niobium, molybdenum, technetium, ruthenium, rhodium, palladium, silver, lanthanum, hafnium, tantalum, tungsten, rhenium, osmium, iridium, platinum, gold and mercury.

In particular embodiments of the method according to the invention, the transition metal present in the culture medium is chosen from the transition metals of group 10, of group 11, or of group 12 of the periodic table of the elements.

The culture medium may contain, as element in excess, a single element chosen from the transition metals, lead and selenium, or a plurality of such elements.

Defensins are well known per se. They are small cationic proteins synthesized by invertebrates, vertebrates, plants and fungi, which are involved in the innate immune defense system (Ganz et al., 1995; Boman, 1998). Several biological activities of defensins have been described in the literature, especially antibacterial and antifungal activities (Ganz et al., 1995; Lay et al., 2005), as has the ability of certain defensins to confer zinc tolerance on plants and yeasts (Mirouze et al., 2006; Shahzad et al., 2013).

Plant defensins in particular are rich in cysteines and have a globular three-dimensional structure stabilized by four disulfide bridges. They share a characteristic motif, the CSαβ motif, which is composed of an a helix and two or three antiparallel β sheets stabilized by three or four disulfide bridges, and/or what is referred to as a gamma-core loop, characterized by a GXC-X₃₋₉-C motif, which may be located between the second and third β sheets (Cornet et al., 1995; Lay et al., 2005, Van der Weerden et al., 2013a, 2013b; Munoz et al., 2014).

In preferred embodiments of the invention, the eukaryotic cells subjected to the culturing step are modified to express or overexpress a defensin of plant origin.

According to the present invention, the eukaryotic cells subjected to the culturing step are preferably modified to express or overexpress a defensin:
of plant origin encoded by a gene of the Plant Defensin type 1 (PDF1) family, for example the defensin AhPDF1.1b of Arabidopsis halleri of amino acid sequence SEQ ID NO:2 (GenBank accession numbers: HF545648 for the nucleotide sequence and CCN97877 for the protein sequence; Shahzad et al., 2013). This defensin was initially named AhPDF1.1 (Mirouze et al., GenBank accession numbers: AY961376 for the nucleotide sequence and AAY27736 for the protein sequence); and/or
having a three-dimensional structure comprising a CSαβ type motif and/or a gamma-core loop, of plant or other origin. Those skilled in the art will know how to readily identify, from existing defensins, those which have such a CSαβ type motif and/or such a gamma-core loop, from data published in the literature, or by comparison, by molecular modeling, for example with the PyMol software, of their structure with the structure of radish defensin RsAFP1 (PDB accession number: 1AYJ) described in the abovementioned publications; and/or
having at least 30% amino acid identity with a defensin of plant origin encoded by a gene of the Plant Defensin type 1 family, in particular the defensin AhPDF1.1b of Arabidopsis halleri of sequence SEQ ID NO: 2.

The percentage of amino acid identity between two peptide sequences may be determined conventionally per se, by comparing the optimally aligned sequences. Such an optimal alignment may especially be carried out by computer means adapted for this purpose, for example by means of the BLAST software. The differences in amino acids between the sequence tested and the reference sequence (defensin of plant origin encoded by a gene of the Plant Defensin type 1 family, for example AhPDF1.1b) may consist of deletions, insertions and/or substitutions of one or more consecutive or non-consecutive amino acids.

By way of examples, defensins having a three-dimensional structure comprising a CSαβ type motif and/or a gamma-core loop and/or at least 30% amino acid identity with a defensin of plant origin encoded by a gene of the Plant Defensin type 1 family, and which may be used within the context of the invention, are:
the defensin AhPDF1.4 of Arabidopsis halleri, of amino acid sequence SEQ ID NO:4 (GenBank accession number: CCN97876) and of nucleotide sequence SEQ ID NO:3 (GenBank accession number: HF545647). This defensin has a CSαβ type motif and a gamma-core loop. It has 38% amino acid identity with AhPDF1.1b,
the drosophila defensin (drosomycin, GenBank accession number: CAA53267). This defensin has a CSαβ type motif and a gamma-core loop;
the oyster defensin (GenBank accession number: ACQ76274). This defensin has a CSαβ type motif and a gamma-core loop;
the plant defensin of common wheat (Triticum aestivum) TaDEF (GenBank accession numbers: AB089942 for the nucleotide sequence and BAC10287 for the protein sequence). This defensin has a CSαβ type motif and a gamma-core loop (Van der Weerden et al., 2013a). It has 33% amino acid identity with AhPDF1.1b in the mature protein;
the plant defensin of the plant Medicago sativa (GenBank accession numbers: AY681971 for the nucleotide sequence and AAV85436 for the protein sequence). This defensin has a CSαβ type motif and a gamma-core loop (Sagaram et al., 2011). It has 29% amino acid identity with AhPDF1.1b in the mature protein.

The method according to the invention, by deregulation of the cell metabolism of the eukaryotic organism, more particularly of the sterol biosynthesis pathway, advantageously makes it possible to increase the level of production by the cells of any compound of interest synthesized in the sterol biosynthesis pathway. The mechanism giving rise to such an advantageous result will not be prejudged here. However, it has been demonstrated by the present inventors that the expression of a defensin in the cell, combined with presenting an excess of at least one element chosen from the transition metals, lead and selenium in the culture medium, activates the vast majority of the genes involved in the sterol biosynthesis pathway, leading to a highly increased production, successively, of each of the intermediate compounds synthesized in this pathway, and leading to a very high accumulation of the final compound.

Such a method is advantageously simple and inexpensive to carry out, and in particular it makes it possible to obtain a compound of interest with a very high level of purity and also a very high level of production. It therefore constitutes a highly advantageous alternative route to producing sterols.

In particular embodiments of the invention, the element chosen from the transition metals, lead and selenium is present in the culture medium at a concentration greater than or equal to the concentration of this element which is necessary to induce the production by the cells of an amount of the compound of interest, in particular of said compound of the sterol biosynthesis pathway, at least 5 times, preferably at least 10 times, and for example at least 50 times greater than the amount produced by the cells cultured under identical culture conditions, the culture medium however containing the element at the minimum concentration necessary for the optimal growth of the cells.

In particular embodiments of the invention, the eukaryotic organism is chosen from yeasts, fungi, algae and microalgae, plants and animals.

When the eukaryotic organism is of the type which does not naturally produce defensin, and in particular not a particular defensin as defined above, for example when this organism is a yeast, the cells of said organism subjected to the culturing step in accordance with the present invention are modified so as to be able to produce such a defensin.

When the eukaryotic organism is of the type which does naturally produce a defensin, for example a plant organism, the cells of this organism subjected to the culturing step in accordance with the present invention may be modified so as to be able to overexpress a defensin. This defensin may be the defensin that they produce naturally or another defensin, especially a particular defensin as defined above.

Thus, in particular embodiments of the invention, the cells of the eukaryotic organism are recombinant cells into which a nucleotide sequence, coding for the defensin to be expressed or overexpressed, has been introduced.

Such a modification of the cells may be carried out by any conventional method per se and known to those skilled in the art, for example by transformation of the strain with an appropriate expression vector comprising one or more genes coding for the defensin or a precursor thereof. Each of such genes may be placed under the control either of a constitutive promoter or an inducible promoter.

The cells may especially be yeast cells, for example of Saccharomyces cerevisiae, Pichia pastoris or Yarrowia lipolytica, genetically modified to express a plant defensin, in particular a defensin encoded by a gene of the PDF1 family, for example AhPDF1.1b.

In particular embodiments of the invention, particularly suited to the cases in which the eukaryotic organism is a yeast, and in which the element must be present in the culture medium to ensure growth of the cells, in particular when this element is zinc, the concentration in the culture medium of the element chosen from the above-mentioned list is at least equal to 1 mM, preferably at least equal to 5 mM, and preferentially at least equal to 10 mM. This concentration is especially between 15 and 30 mM, for example approximately equal to 20 mM. Such a concentration advantageously makes it possible to obtain the highest levels of activation of the sterol biosynthesis pathway, in particular when the element contained in the culture medium is zinc and the culture medium is the customarily used medium Yeast Nitrogen B Base (DIFCO).

In different embodiments of the invention, particularly suited to the cases in which the element is not necessary for growth of the cells, for example when this element is cadmium, the concentration in the culture medium of the element chosen from the above-mentioned list may be for example between 10 and 100 μM.

In particular embodiments of the invention, the method comprises a prior step of in vitro culture of the cells in a culture medium adapted to the growth of the cells and containing the above-mentioned elements, that is to say the transition metals, selenium and lead, at the minimum concentrations thereof necessary for growth of the cells. This step, which is referred to as pre-culturing, may be carried out in the same culture medium as the culturing step in the presence of an excess of at least one of said elements, or in a different medium.

The method according to the invention thus advantageously makes it possible to decouple the production of biomass and the production of a compound of interest produced in the sterol biosynthesis pathway. Indeed, it thus makes it possible to produce, in a prior step, a large amount of biomass, then in a second step to trigger the activation of the sterol biosynthesis pathway, especially by simply adding a larger amount of the element into the culture medium, so as to produce the targeted compound of interest in large amounts.

The method according to the invention thus also constitutes a method for increasing the level of production, by a eukaryotic organism, of a compound of interest that can be synthesized by this organism in this biosynthesis pathway, or derived from such a compound.

The method according to the invention preferably comprises a subsequent step of collecting the cells of the eukaryotic organism having accumulated the compound of interest, and, where appropriate, of extracting the compound of interest.

The compound of interest having been produced in large amounts by the cells may be extracted according to any conventional technique per se, especially starting from a cell lysate or culture supernatant, depending on the given compound. This extraction may comprise a step of purification of the compound of interest produced, for example by crystallization or by a chromatography technique such as affinity chromatography or high-performance liquid chromatography (HPLC) etc.

In particular embodiments of the invention, the compound of interest is a final compound in the sterol biosynthesis pathway in the eukaryotic organism in question, especially a sterol. For example, when the organism is a yeast, the compound of interest may be ergosterol.

The compound of interest produced may otherwise be an intermediate compound in the sterol biosynthesis pathway.

The cells of the eukaryotic organism subjected to the culturing step in the presence of said at least one element may then be recombinant cells which are modified according to the invention to inhibit an enzyme activity involved in the biotransformation of this intermediate compound in the sterol biosynthesis pathway, especially to inhibit the expression of a gene coding for such an enzyme activity. Such a modification of the cells makes it possible to block the sterol biosynthesis pathway downstream of the intermediate compound of interest, the production of which is desired, in order to avoid the biotransformation of the latter and in order to consequently cause the accumulation thereof in the cells. This modification may be carried out by any method known in the field of genetic engineering, especially by mutagenesis.

Otherwise, when the compound of interest is an intermediate compound in the sterol biosynthesis pathway, the cells of the eukaryotic organism expressing or overexpressing a defensin may be brought into the presence, in the culture medium, of a reagent having an inhibitory effect on an enzyme activity involved in the biotransformation of said intermediate compound in the sterol biosynthesis pathway. Such an inhibitory effect may apply at various levels in the sterol biosynthesis pathway and is well documented in the prior art, for example in the publication by Burden et al., 1989. In most cases, this type of inhibitor acts directly on the enzyme involved, without the corresponding gene expression being affected. For example, terbinafine is an inhibitor of squalene epoxidase, the action of which causes the accumulation of squalene in yeasts such as S. cerevisiae, as described especially in the publication by Nowosielski et al., 2011.

By way of example, in yeast, the method according to the invention may be used to produce large amounts of the intermediate compounds of the sterol biosynthesis pathway having a particular chemical and/or economic benefit, for example farnesyl diphosphate (FPP), which makes it possible to obtain isoprenoids and terpenes, including carotenoids; squalene, used for example as medical adjuvant, as antioxidant or in cosmetics; lanosterol, a component of lanolin, which can be used in cosmetics, in pharmaceuticals or for leather treatment, for example.

When the eukaryotic organism is a yeast, the cells of the eukaryotic organism may for example be modified to inhibit the expression of a gene chosen from ERG1, associated with the enzyme activity of squalene biotransformation; NCP1, also associated with the enzyme activity of squalene biotransformation; ERG11, associated with the enzyme activity of lanosterol biotransformation; ERG9, associated with the enzyme activity of FPP biotransformation in the sterol biosynthesis pathway; ERG2, associated with the enzyme activity of fecosterol biotransformation; or else ERG3, associated with the enzyme activity of episterol biotransformation.

Eukaryotic strains, in particular of yeasts, modified to inhibit an enzyme activity involved in the biotransformation of an intermediate compound in the sterol biosynthesis pathway into another compound in the sterol biosynthesis pathway have been developed by genetic engineering techniques and described in the prior art. By way of example, an ERG2 mutant and an ERG3 mutant, enabling the accumulation of fecosterol and episterol, respectively, are available from the EUROSCARF Collection Center (Frankfurt) under the accession numbers Y00788 and Y02667, respectively.

In variants of the invention, the compound of interest is a compound derived from a compound of the sterol biosynthesis pathway, for example derived from a final compound of this pathway, or derived from an intermediate compound of this pathway. The cells of the eukaryotic organism subjected to the culturing step in the presence of said at least one element are thus recombinant cells which are modified to activate at least one, or even several, enzyme activity/activities involved in the biotransformation of said compound of the sterol biosynthesis pathway into said compound of interest. Such an enzyme activity may especially be activated by modifying the cells by introducing a nucleotide sequence coding for an enzyme having said enzyme activity, so as to cause the expression of this enzyme in the cells. Otherwise, activation may be carried out by modifying the cells to cause the overexpression of an enzyme naturally produced in the cells and having said enzyme activity.

Where appropriate, when the compound of interest is a compound derived from an intermediate compound of the sterol biosynthesis pathway, said eukaryotic cells may on the one hand be modified to activate said enzyme activity or activities involved in the biotransformation of said compound of the sterol biosynthesis pathway into said compound of interest, and on the other hand modified to inhibit an enzyme activity involved in the biotransformation of said intermediate compound in the sterol biosynthesis pathway, especially to inhibit the expression of a gene coding for such an enzyme activity, as described above.

Numerous eukaryotic strains, in particular of yeasts, modified to activate an enzyme activity involved in the biotransformation of a compound of the sterol biosynthesis pathway into another compound of interest distinct from this pathway have been developed by genetic engineering techniques and described in the prior art.

Thus, by way of nonlimiting examples of the invention, the cells of the eukaryotic organism subjected to the culturing step in the presence of an excess of said at least one element may be recombinant yeast cells modified to activate:
a C-methyltransferase activity so as to enable the overproduction by said cells of 24-ethylsterol and of 24-ethylidenesterol from ergosterol, as described in the publication by Husselstein et al., 1996; or
an enzyme activity for hydroxylation of ergosterol, as described especially in the document WO-A-2011/067144.

According to another example, the cells of the eukaryotic organism subjected to the culturing step in the presence of an excess of said at least one element may be recombinant yeast cells modified in order on the one hand to inhibit a Δ22-desaturase activity and on the other hand to activate Δ7-reductase and adrenodoxin reductase activities, so as to make it possible to overproduce, in the presence moreover of defensin and of an excess of the element in the culture medium, pregnenolone, or optionally progesterone by simultaneous activation of a β-hydroxysteroid dehydrogenase/isomerase activity. Yeast cells modified in this way, but nonetheless not expressing defensin, are described in the publication by Duport et al., 1998.

The method according to the invention may otherwise be used to modify the composition of an oil that can be extracted from a eukaryotic organism, by increasing or reducing the proportion of a particular compound of interest present in this oil. It is thus advantageously applicable in the field of food oils or industrial oils, or in that of biofuels, which it especially makes it possible to enrich in a given compound.

Another aspect of the invention is the combined use of eukaryotic cells modified to express or overexpress a defensin, especially a defensin as described above, and of the presence in excess in the medium for culturing these cells of an element chosen from the transition metals, for example zinc, cadmium, nickel, cobalt and copper, selenium and lead, for the production of a compound of interest of the sterol biosynthesis pathway in said eukaryotic cells, or derived from a compound of this biosynthesis pathway. Such a use aims in particular at producing ergosterol with an advantageously high production yield. The culture conditions may correspond to one or more of the characteristics described above.

According to another aspect, the invention relates to the use of a recombinant yeast strain expressing a defensin for the production, with an increased yield, of a compound of interest of the sterol biosynthesis pathway or derived from a compound of the sterol biosynthesis pathway in yeast.

In particular, cells of this recombinant yeast strain are subjected to a step of in vitro culture in a culture medium adapted to the growth of said cells and containing at least one element chosen from the transition metals, for example zinc, cadmium, nickel, cobalt and copper, lead and selenium, at a concentration greater than or equal to the concentration of said element which is necessary to induce the overproduction of said compound of interest, in particular of said compound of the sterol biosynthesis pathway, by said cells. Overproduction is intended to mean the production by said cells of an amount of said compound of interest, in particular of said compound of the sterol biosynthesis pathway, at least 2 times greater than the amount produced by said cells under identical culture conditions, with the only difference being that the culture medium contains said element at the minimum concentration necessary for the optimal growth of said cells.

The defensin may especially be a defensin:
of plant origin encoded by a gene of the Plant Defensin type 1 family, and/or
having a three-dimensional structure comprising a CSαβ type motif and/or a gamma-core loop, and/or
having at least 30% amino acid identity with the defensin AhPDF1.1b of Arabidopsis halleri of sequence SEQ ID NO: 2.

The conditions for culture of the cells may in particular correspond to one or more of the characteristics described above.

Another subject of the present invention is a recombinant eukaryotic cell modified to express or overexpress a defensin, in particular a defensin as described above, and modified to inhibit an enzyme activity involved in the biotransformation of an intermediate compound of the sterol biosynthesis pathway in said cell, especially to inhibit the expression of a gene coding for such an enzyme activity.

For example, this eukaryotic cell may be modified so as to inhibit the expression of a gene chosen from ERG1, NCP1, ERG2, ERG3, ERG11 and ERG9.

Such an inhibition may be carried out by any conventional method per se for those skilled in the art. For example, it may be carried out by genetic engineering techniques, by establishing mutants referred to as “knockout” by insertion of a recombinant DNA in the gene concerned, or else by genetic mutagenesis.

This eukaryotic cell may also be further modified, to activate an enzyme activity involved in the transformation of this intermediate compound into a compound of interest.

According to another subject, the present invention relates to a recombinant eukaryotic cell modified to express or overexpress a defensin, in particular a defensin as described above, and modified to activate an enzyme activity involved in the transformation of a compound, especially a final compound, of the sterol biosynthesis pathway, into a compound of interest distinct from this pathway.

Such cells according to the invention may especially be yeast cells, in particular of the species Saccharomyces cerevisiae, Pichia pastoris or Yarrowia lipolytica, expressing a plant defensin, in particular of the type encoded by a gene of the PDF1 family, for example the defensin AhPDF1.1b.

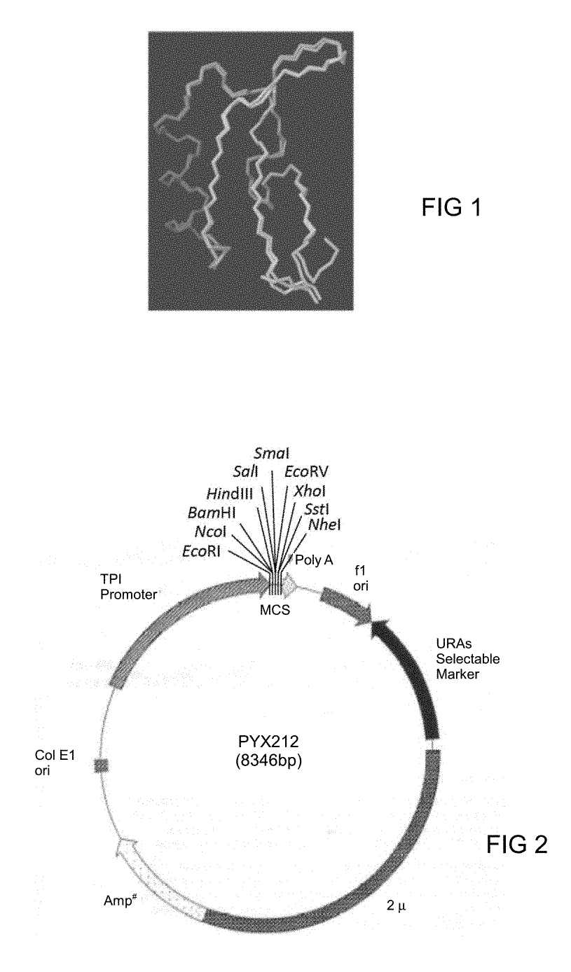

The features and advantages of the invention will become more clearly apparent in light of the exemplary embodiment below, provided simply by way of nonlimiting illustration of the invention, with the support of FIGS. 1 to 6, in which:

FIG. 1 shows the superposition of the structures obtained by molecular modeling of the defensin AhPDF1.1b and of the reference radish defensin RsAFP1, respectively;

FIG. 2 shows the map of the empty pYX212 vector;

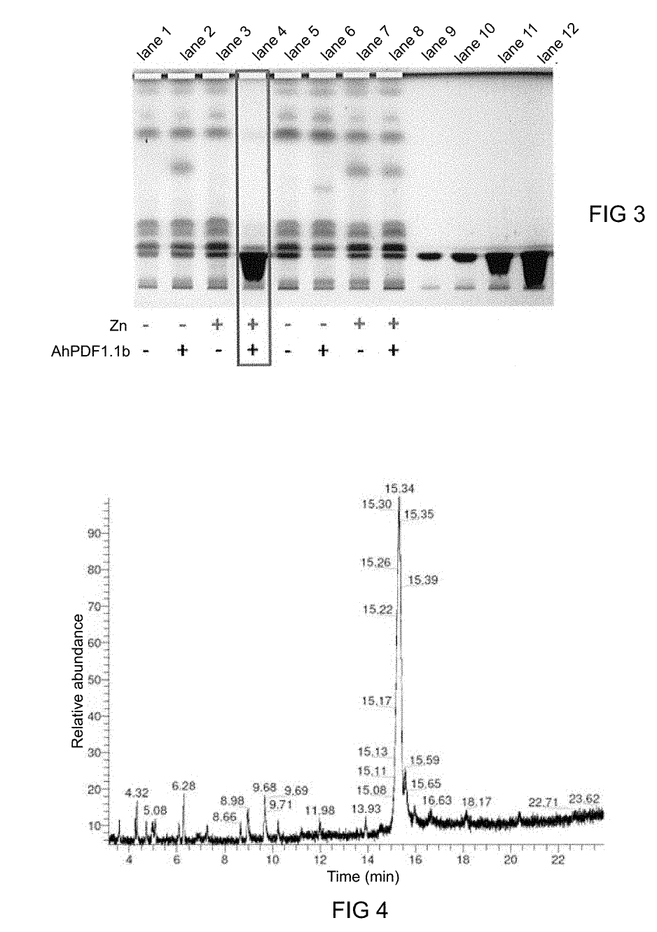

FIG. 3 shows a plate obtained by thin-layer chromatography (TLC) for the fractions of neutral unsaponifiable lipids (lanes 1 to 4) and of other unsaponifiable lipids (lanes 5 to 8), respectively, extracted from cells following culture of the BY4741 strain of Saccharomyces cerevisiae expressing (AhPDF1.1b+) or not expressing (AhPDF1.1b−) the plant defensin AhPDF1.1b, in the presence of zinc in excess at 20 mM (Zn+) or of zinc at 2.5 μM (Zn−) in the culture medium; lanes 9 to 12 correspond to deposits of pure ergosterol, in the amounts 5 μg (lane 9), 10 μg (lane 10), 25 μg (lane 11), and 50 μg (lane 12), respectively;

FIG. 4 shows a chromatogram obtained by gas chromatography for the product obtained by preparative TLC from the fraction of neutral unsaponifiable lipids extracted from the cells after culturing the BY4741 strain of Saccharomyces cerevisiae expressing the plant defensin AhPDF1.1b, in the presence of 20 mM of zinc in the culture medium;

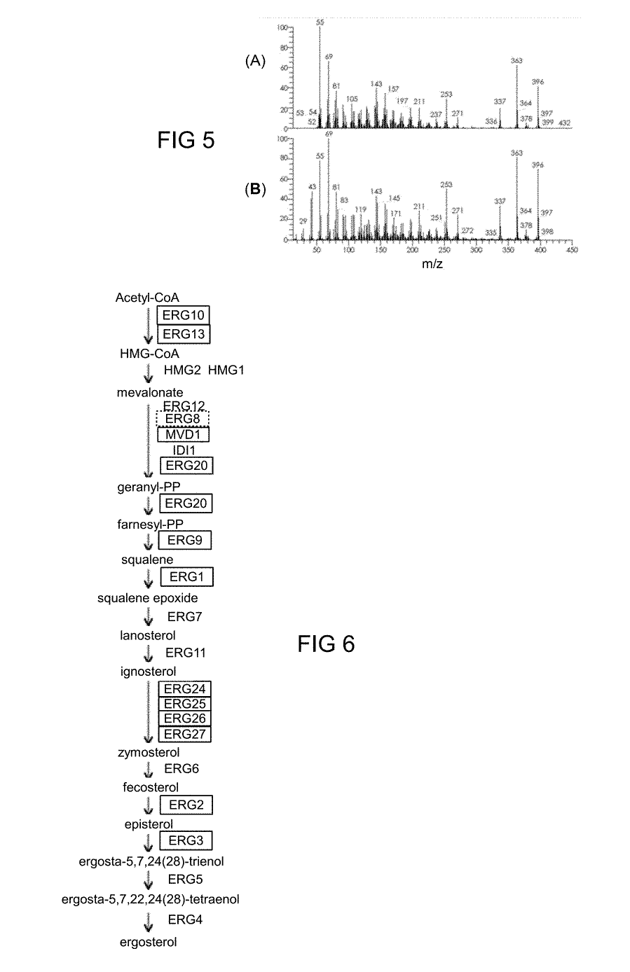

FIG. 5 shows the mass spectra obtained for (A) the peak at 15.34 min retention time from the chromatogram of FIG. 4, and (B) pure ergosterol; and

FIG. 6 illustrates the projection, on the biosynthesis pathway of ergosterol in yeast, of the results of the differential expression of the genes involved in this biosynthesis pathway between the two following conditions: recombinant yeast expressing the defensin AhPDF1.1b and subjected to a treatment with zinc (20 mM), and control yeast (no defensin) cultured under the same conditions; the genes with a significantly higher amount of transcripts in the yeasts expressing defensin are shown in boxes with a solid outline, and the gene with a significantly lower amount of transcripts in the yeasts expressing defensin is shown in a box with a dashed outline.

### EXAMPLE 1—DEFENSIN AhPDF1.1b AND EXCESS OF ZINC

A strain of Saccharomyces cerevisiae was modified to express the plant defensin of Arabidopsis halleri AhPDF1.1b, then cultured in the presence of an excess of zinc in accordance with the present invention, in the following way.

The defensin AhPDF1.1b of Arabidopsis halleri (Genbank accession number: HF545648, nucleotide sequence SEQ ID NO:1, amino acid sequence SEQ ID NO:2) was firstly the subject of a molecular modeling study (Swiss Model, Geno3D or PyMol) and its structure was superimposed onto that of radish defensin RsAFP1 (amino acid sequence: GenBank accession number AAA69541; nucleotide sequence: GenBank accession number U18557) which has been widely described in the literature (Terras et al., 1995) as having a structure with a CSαβ type motif and a gamma-core loop. The representation obtained is shown in FIG. 1. A perfect superposition of the two defensins is observed here, demonstrating that the defensin AhPDF1.1b of Arabidopsis halleri has a CSαβ type motif and gamma-core loop structure.

The defensin AhPDF1.1b coding sequence was amplified by polymerase chain reaction (PCR) and cloned into the vector pYX212 (sold by Ingenenius), the map of which is shown in FIG. 2, at the restriction sites EcoRI and XhoI.

The coding sequence is under the control of the yeast triose phosphate isomerase (TPI) constitutive promoter and upstream of the yeast heat shock protein HSF1 terminator (YGL073w).

The recombinant plasmid obtained, and also the empty plasmid pFL38H carrying the gene HIS3 which confers auxotrophy to histidine, described in the publication by Talke et al., 2006, were introduced into the yeast strain BY4741 (Mat a, his3Δ1, leu2Δ0, met15Δ0, ura3Δ0) using the method for transformation using PEG and lithium chloride (Gietz & Woods, 2002). The strain BY4741 was transformed in parallel with the empty plasmids pYX212 and pFL38H and serves as negative control.

The recombinant strains were cultured at 30° C. at the density of approximately 50 CFU/cm² on solid medium (1.43 g/l of Yeast Nitrogen Base without amino acids or ammonium (Ref. 233520, Difco), 20 g/l glucose, 6.4 g/l NH₄NO₃, 50 mM succinic acid-KOH pH 4.5, methionine 20 mg/l, leucine 60 mg/l, 20 g/l agarose (ref. D5, Euromedex)), supplemented:
either by zinc sulfate (ZnSO₄) at the final concentration of 2.5 μM, which corresponds to the minimum concentration necessary for the optimal growth of the cells, or
by zinc sulfate (ZnSO₄) at the final concentration of 20 mM, that is to say in excess in accordance with the present invention.

For each experiment, when the diameter of the colonies reached 0.48±0.02 mm on average, which corresponds to an exponential growth phase, the yeasts were harvested, rinsed twice in pure water and then lyophilized.

The neutral lipids were extracted with hexane and the remaining (polar) lipids were extracted with a mixture of chloroform/methanol (2V/1V) according to the protocol described by Lomascolo et al., 1994.

Each of the lipid fractions was then saponified according to standard AFNOR NFT 60-205 and samples of the unsaponifiable fractions containing between 50 and 100 μg of lipids were taken off.

The samples were analyzed by thin-layer chromatography (TLC) on silica gel TLC plate (ref. 60 F 254 from E. Merck KGaA).

As controls, different amounts (respectively five, 10, 25 and 50 μg) of pure ergosterol (Ref. 45480, Sigma) were deposited in parallel. Migration was carried out in a buffer hexane:ether:formic acid in the proportions 70:30:1. At the end of migration, the plate was air-dried then sprayed uniformly with a 50:50 solution of pure orthophosphoric acid and saturated copper sulfate. After drying, the plate was heated at 180° C. for 8 to 10 min. The amounts of sterols were determined by comparison with the samples constituting the calibration range, by means of a densitometer (CAMAG TLC Scanner 3 model “Scanner3_160813” S/N 160813 (1.14.28), E. Merck KGaA) which analyzes the plate at the wavelength of 325 nm.

The results obtained are shown in FIG. 3. As can be seen, the cells expressing defensin and cultured in the presence of zinc at 20 mM in the culture medium, and only these cells, lead to obtaining, in the unsaponifiable neutral lipid fraction, a very large amount of a particular compound, the characteristic band of which can clearly be identified as corresponding to ergosterol, by comparison with the lanes containing pure ergosterol, of general formula (I):

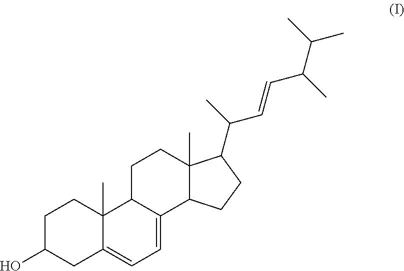

Ergosterol is considerably predominant in this fraction. It can be established that it represents more than 80% thereof.

The confirmation that this particular compound is indeed ergosterol was established by gas chromatography analysis coupled with mass spectrometry (GC/MS) in the following way.

A preparative TLC was carried out starting from 100 μL of the neutral unsaponifiable lipid fraction obtained for the culture condition expressing the plant defensin AhPDF1.1b, in the presence of 20 mM of zinc in the culture medium. After migration, the plate was scraped at the mark identified as corresponding to ergosterol. The product was extracted from the silica with chloroform. After filtration to totally eliminate the silica particles, the chloroform was evaporated and the extract was redissolved in hexane before being injected into gas chromatography coupled with a mass spectrometer.

The chromatogram obtained is shown in FIG. 4 (total ionic abundance in the m/z range of 50 to 450). A considerably predominant peak is observed there, eluted after 15.34 min, which corresponds to the expected retention time for ergosterol.

Moreover, the mass spectrum determined for this peak indicates unambiguously that it is ergosterol, as shown in FIG. 5, in which a correlation between the mass spectra (A) of the peak at 15.34 min and (B) of pure ergosterol is observed.

The production yields of ergosterol by the yeasts, expressed in mg per g of dry weight of yeasts (mg/gDW) were moreover determined for each of the culture conditions. The results are indicated in table 1 below.

TABLE 1
production yields of ergosterol by yeasts as a function of the
transformation/culture conditions

| Zinc concentration | | Defensin AhPDF1.1b | Yield (mg/gDW) |
|---|---|---|---|
| 2.5 | μM | No expression | 4 |
| 2.5 | μM | Expression | 4 |
| 20 | mM | No expression | 5 |
| 20 | mM | Expression | >120 |

For the combination “20 mM of zinc—expression of defensin”, the ergosterol production yield is considerably greater than that obtained for the other conditions.

The sterols represent more than 12% by weight relative to the total dry weight of yeasts.

Which genes of the sterol biosynthesis pathway are affected by the activation process according to the invention was also studied.

To this end, a transcriptomic approach was used.

The same yeast strains as those described above were cultured in the same conditions as described above. When the diameter of the colonies reached an average of 0.48±0.02 mm, the yeasts were harvested and immediately frozen in liquid nitrogen.

The RNA was then extracted using the method with TRIZOL®, described in the publication by Chomczynski and Sacchi, 1987. Briefly, the cells were milled by intense agitation in the presence of 0.3 mm glass beads in Trizol at 4° C. for 15 min. Chloroform was added at an amount of 1/5 of the volume. After centrifugation for 15 min at 9000×g, the supernatant was recovered and isopropyl alcohol was added at an amount of 50% of the volume of supernatant. After 10 min of incubation at −20° C. then 10 min of centrifugation at 9000×g, the pellet was recovered and rinsed twice in 75% ethanol then taken up again in 150 μl of water.

100 μg of total RNA were then purified using the RNeasy kit (Qiagen). The DNA was eliminated by using a DNase directly on the kit column. The RNA was eluted in 30 μl of water.

100 ng of purified RNA were labelled with cyanin3-dCTP using the “Quick Amp Labeling one-color kit” from Agilent Technologies. The labeled RNAs were hybridized on a yeast DNA chip “8-15 15 karray Agilent standard Yeast V2 g-Gene Expression Microarray” (Agilent Technologies) for 17 h at 65° C. After rinsing, the slides were read on a GENEPIX® 4000B scanner.

From the datasheet giving the hybridization intensities for all the genes under the four conditions tested (control yeasts (“Def−”) and yeasts expressing defensin (“Def+”) cultured in the presence of zinc at the final concentration of 2.5 μM (“Zn−”) or of zinc at the final concentration of 20 mM (“Zn+”)), the results obtained relating to the expression of all the genes involved in the ergosterol biosynthesis pathway are given in table 2 below.

TABLE 2
Expression of the genes involved in the ergosterol biosynthesis
pathway as a function of the transformation/culture conditions
(presence (“Def+”) or absence (“Def−”) of defensin expression,
in the presence of zinc at 20 mM (“Zn+”) or zinc at 2.5 μM
(“Zn−”) in the culture medium)

| Gene | Def−/Zn− | Def+/Zn− | Def−/Zn+ | Def+/Zn+ |
|---|---|---|---|---|
| ERG1 | 10.063 | 10.011 | 8.8487 | 9.5168 |
| ERG2 | 10.666 | 10.483 | 9.9281 | 10.286 |
| ERG3 | 11.533 | 11.444 | 11.790 | 12.830 |
| ERG4 | 10.527 | 10.377 | 10.816 | 11.373 |
| ERG5 | 9.0856 | 9.1176 | 9.2295 | 9.4736 |
| ERG6 | 9.1971 | 8.7485 | 8.9209 | 9.1201 |
| ERG7 | 9.9551 | 10.065 | 9.4971 | 10.002 |
| ERG8 | 10.597 | 10.686 | 11.022 | 10.367 |
| ERG9 | 10.373 | 10.337 | 10.245 | 10.733 |
| ERG10 | 10.835 | 10.742 | 9.7926 | 10.790 |
| ERG11 | 11.167 | 11.170 | 10.396 | 11.028 |
| ERG12 | 8.0901 | 7.8844 | 8.0822 | 8.0168 |
| ERG13 | 9.5652 | 9.4018 | 8.9288 | 9.4047 |
| ERG20 | 11.760 | 11.716 | 10.034 | 11.120 |
| ERG24 | 10.013 | 9.9343 | 9.1541 | 9.8254 |
| ERG25 | 11.768 | 11.733 | 11.603 | 12.798 |
| ERG26 | 9.8690 | 9.7937 | 9.4158 | 9.9413 |
| ERG27 | 10.072 | 10.279 | 9.6617 | 10.612 |
| ERG28 | 9.4582 | 9.4305 | 8.6241 | 9.4682 |
| HMG1 | 8.9564 | 8.9347 | 8.8616 | 9.0901 |
| HMG2 | 10.308 | 10.321 | 9.8840 | 10.198 |
| IDI1 | 10.804 | 10.842 | 10.539 | 10.394 |
| MVD1 | 8.2992 | 8.3027 | 8.0140 | 8.6319 |

It is observed in particular that the expression of the genes ERG1, ERG3, ERG4, ERG7, ERG9, ERG10, ERG11, ERG13, ERG20, ERG24, ERG25, ERG26, ERG27, ERG28, MVD1 is increased, sometimes greatly, for the combination “expression of defensin+addition of zinc in excess (20 mM) in the culture medium” compared to the condition “normal yeast+addition of zinc in excess in the culture medium”; and as regards the genes ERG3, ERG4, ERG9, ERG25, ERG27 and MVD1, compared to all the conditions tested.

The results obtained above are projected onto the ergosterol biosynthesis pathway in yeast, comparing the condition “expression of defensin/presence of zinc in excess in the culture medium” (“Def+/Zn+”) with the condition “absence of defensin/presence of zinc in excess in the culture medium” (“Def−/Zn+”). The result is shown in FIG. 6. In this figure, the box with a dashed outline surrounding the name of the gene indicates a reduction in the expression of the gene in the yeast expressing defensin, and the box with a solid outline indicates an increase in the expression of the gene in the yeast expressing defensin.

### EXAMPLE 2—DEFENSIN AhPDF1.4 AND EXCESS OF ZINC

A strain of Saccharomyces cerevisiae was modified to express the plant defensin of Arabidopsis halleri AhPDF1.4 (nucleotide sequence SEQ ID NO:3, amino acid sequence SEQ ID NO:4; this defensin has a CSαβ type motif and a gamma-core loop, and has 38% amino acid identity with AhPDF1.1b), then cultured in the presence of an excess of zinc (20 mM), in accordance with the present invention, following the same protocol as that described in example 1 above. By way of comparison, a culture in the same culture medium containing zinc at a much lower concentration (2.5 μM), which is not considered as an excess within the context of the present invention, was also carried out.

The yields of production of ergosterol by yeasts, expressed in mg per g of dry weight of yeasts (mg/gDW) were determined for each of the culture conditions. The results are indicated in table 3 below.

TABLE 3
Yields of production of ergosterol by yeasts as a function of the
transformation/culture conditions

| Zinc concentration | | Defensin AhPDF1.4 | Yield (mg/gDW) |
|---|---|---|---|
| 2.5 | μM | No expression | 2.5 |
| 2.5 | μM | Expression | 1.5 |
| 20 | mM | No expression | 1 |
| 20 | mM | Expression | 41.5 |

In this case, too, the yield of production of ergosterol is considerably increased for the combination “excess of zinc in the culture medium—expression of defensin”.

### EXAMPLE 3—DEFENSINS TaDEF and MsDEF1 AND EXCESS OF ZINC

Strains of Saccharomyces cerevisiae were modified, according to the method described in example 1 above, to express, respectively:
the plant defensin of common wheat Triticum aestivum TaDEF (nucleotide sequence SEQ ID NO:5, GenBank accession number AB089942; amino acid sequence SEQ ID NO:6, GenBank accession number BAC10287, this defensin has a CSαβ type motif and a gamma-core loop, and has 33% amino acid identity with AhPDF1.1b in the mature protein); or
the plant defensin of the Medicago sativa plant MsDEF1 (nucleotide sequence SEQ ID NO:7, GenBank accession number AY681971; amino acid sequence SEQ ID NO:8, GenBank accession number AAV85436; this defensin has a CSαβ type motif and a gamma-core loop, and has 29% amino acid identity with AhPDF1.1b in the mature protein).

The strains modified in this way were cultured in the presence of an excess of zinc (20 mM), in accordance with the present invention, following the same protocol as that described in example 1 above.

By way of comparison, a strain of Saccharomyces cerevisiae modified to express the defensin AhPDF1.1b was also cultured in the same culture medium containing an excess of zinc (20 mM).

For each experiment, when the diameter of the colonies has reached 0.48±0.02 mm on average, which corresponds to an exponential growth phase, the yeasts were harvested, rinsed twice in pure water and then lyophilized.

The lyophilized samples were subjected to a step of saponification before extraction and analysis. For this purpose, a few tens of mg of sample had added to them 400 μl of a 10% (w/v) methanolic KOH solution, 0.2 ml of microbeads 0.1 mm in diameter and 6 mg of 7-dihydrocholesterol (7-DHC) used as internal standard. The mixture was then incubated at 85° C. for 2 hours with vigorous stirring. After cooling to room temperature, 300 μl of water were added. The samples were extracted three times with 700 μl of hexane. The hexane phases containing the unsaponifiable lipids were brought back together and dried, and then taken up in 200 μl of chloroform.

A Shimatzu GC 2010 Plus gas chromatograph, fitted with an automatic injector, a ZEBRON® ZB-5HT INFERNO® capillary column and a flame ionization detector, was used to assay the ergosterol, with reference to a standard range of commercial ergosterol (Sigma, purity ≧95%) and to an internal standard (7-DHC). The volume injected was 0.2 μl with a ratio of division of 1/25. The temperature of the injector and the detector was 310° C. That of the oven was made to vary from 270° C. to 310° C. in 10 min, then maintained at 310° C. for 10 min.

The yields of production of ergosterol by yeasts, expressed in μg per g of dry weight of yeasts (mg/gDW) were determined for each of the culture conditions. The results are indicated in table 4 below.

TABLE 4
Yields of production of ergosterol by yeasts as a function
of the defensin employed (in the presence of 20 mM of zinc)

| Defensin | Yield (mg/gDW) |
|---|---|
| Control without defensin | 2.0 |
| AhPDF1.1b | 13.7 |
| TaDEF | 6.5 |
| MsDEF | 10.5 |

As can be observed, in the presence of an excess of zinc (20 mM), the yeasts which express the defensins TaDEF and MsDEF have an increase in the ergosterol content by a factor of greater than 3 and greater than 5, respectively, compared to the yeasts which do not express defensin.

These defensins TaDEF and MsDEF also enable, as does AhPDF1.1b, the overproduction of ergosterol by yeasts.

### EXAMPLE 4—DEFENSIN AhPDF1.1b AND EXCESS OF CADMIUM

A strain of Saccharomyces cerevisiae was modified to express the plant defensin of Arabidopsis thaliana AhPDF1.1b then cultured in the presence of an excess of cadmium (10 or 20 μM) following the same protocol as that described in example 1 above.

The protocol for saponification, for extraction of the unsaponifiables and the GC FID analysis is the same as for example 3 presented above.

The yields of production of ergosterol by yeasts, expressed in μg per g of dry weight of yeasts (μg/gDW) were determined for each of the culture conditions. The results are indicated in table 5 below.

TABLE 5
Yields of production of ergosterol by yeasts

| Cadmium concentration | Expression of | |
|---|---|---|
| (μM) | AhPDF1.1b | Yield (μg/gDW) |
| 10 | No expression | 21.3 |
| 10 | Expression | 43.7 |
| 20 | No expression | 35.6 |
| 20 | Expression | 91.5 |

It is observed that under the conditions of expression of defensin and in the presence of cadmium, the production of ergosterol is significantly increased compared to the other conditions, this effect being more pronounced at a concentration of cadmium in the culture medium of 20 μM.

The defensin AhPDF1.1b is thus capable of inducing an increase in the production of ergosterol when an excess of cadmium is provided in the medium.

### EXAMPLE 5—OVERPRODUCTION OF INTERMEDIATE COMPOUNDS OF THE STEROL BIOSYNTHESIS PATHWAY IN YEAST

The following strains of Saccharomyces cerevisiae were used:
mutant ERG2 (genotype BY4741; Mat a; his3Δ1, leu2Δ0, met15Δ0, ura3Δ0, YMR202w::kanMX4), available from the EUROSCARF collection center (http://web.uni-frankfurt.de/fb15/mikro/euroscarf/about.html) under the accession number Y00788. In this mutant the sterol biosynthesis pathway is blocked to enable the accumulation of fecosterol, by inhibition of the enzyme activity associated with the gene ERG2;
mutant ERG3 (genotype BY4741; Mat a; his3Δ1, leu2Δ0, met15Δ0, ura3Δ0, YLR056w::kanMX4), available from the EUROSCARF collection center (http://web.uni-frankfurt.de/fb15/mikro/euroscarf/about.html) under the accession number Y02667. In this mutant the sterol biosynthesis pathway is blocked to enable the accumulation of episterol, by inhibition of the enzyme activity associated with the gene ERG3;
mutant NCP1 (genotype W303-1B, MATalpha, leu2-3,112 trp1-1 can1-100 ura3-1 ade2-1 his3-11,15 YHR042w::TRP1). In this mutant the sterol biosynthesis pathway is blocked to enable the accumulation of squalene, by inhibition of the enzyme activity associated with the gene NCP1.

Each of these mutants was modified to express the plant defensin of Arabidopsis halleri AhPDF1.1b, then cultured in the presence of an excess of zinc in accordance with the present invention, according to the protocol indicated in example 1 above, to overproduce the associated intermediate compound of interest of the sterol biosynthesis pathway, that is to say fecosterol, episterol and squalene, respectively.

### EXAMPLE 6—OVERPRODUCTION OF COMPOUNDS IN THE MOSS PHYSCOMITRELLA PATENS

The coding sequence of the defensin AhPDF1.1b was introduced into either one of the plasmids proAct1in108Kan and BNRr-108-3′-5′-HSP-CaMV, under the control of the rice constitutive actin promoter and the soy inducible heat shock protein (HSP) promoter, respectively.

The plasmids were integrated at the locus 108 of the genome of the moss Physcomitrella patens by genetic transformation, as described in the publication by Schaefer and Zryd, 1997.

The clones were selected in the presence of kanamycin in the medium (selection marker).

For the overproduction of compounds of the sterol biosynthesis pathway, the clones are cultured in the following culture medium referred to as PPNH₄, enriched with 4 mM of zinc: 3.3 mM Ca(NO₃)₂, 1 mM MgSO₄, 0.5 mM FeSO₄, 0.2 mM KPO₄ pH 7, 0.3 mM ammonium tartrate, 10 μM H₃BO₃, 15 μM MnCl₂, 0.2 μM CuSO₄, 2 μM ZnSO₄, 0.2 μM KI, 0.2 μM CoCl₂, 0.1 μM Na₂MoO₄, 7 g/l agar.

### BIBLIOGRAPHIC REFERENCES

Boman, 1998, Scandinavian Journal of Immunology 48: 15-25
Burden et al., 1989, Phytochemistry, 28: 1791-1804
Burg et al., 2011, Progress in Lipid Research 50: 403-410
Chomczynski et Sacchi, 1987, Anal Biochem. 162:156-159
Cornet et al., 1995, Structure 3: 435-48
Duport et al., 1998, Nature Biotechnology 16: 186-189
Ganz et al., 1995, Pharmacology & Therapeutics 66: 191-205
Gietz & Woods, 2002, Methods in Enzymology 350: 87-96
Husselstein et al., 1996, FEBS Letters 381: 87-92
Kuhn et al., 1979, Eur. J. Appl. Microbiol. Biotechnol. 6: 341-349
Lay et al., 2005, Curr Protein Pept Sci 6: 85-101
Lomascolo et al., 1994, Revue Canadienne de Microbiologie 40: 724-729
Medaglia et Panke, 2010, Appl. Microbiol. Biotechnol. 87: 145-157
Mirouze et al., 2006, The Plant Journal 47: 329-342
Munoz et al., 2014, Mol Microbiol 92: 1357
Nowosielski et al., 2011, Journal of Chemical Information and Modeling 51: 455-462
Sagaram et al., 2011, PLoS One 6(4): e18550
Schaefer et Zryd, 1997, Plant Journal 11: 1195-1206
Shahzad et al., 2013, New Phytologist doi: 10.1111/nph.12396
Shobayashi et al., 2005, Biosci Biotechnol Biochem 69: 2381-2388
Talke et al., 2006, Plant Physiology, 142: 148-167
Terras et al., 1995, Plant Cel. 7(5): 573-588
Van der Weerden et al., 2013a, Fungal Biology Reviews 26: 121-131
Van der Weerden et al., 2013b, Cel. Mol. Life Sci. 70: 3545-3570
Wriessnegger et al., 2013, Progress in Lipid Research 52: 277-293

1. A method for producing a compound of interest of a sterol biosynthesis pathway in a eukaryotic organism or derived from a compound of the sterol biosynthesis pathway in said eukaryotic organism, comprising a step of in vitro culture of cells of said eukaryotic organism, modified to express or overexpress a defensin, in an enriched culture medium configured to grow said cells and containing at least one element chosen from a group consisting of transition metals, lead and selenium, at a concentration greater than or equal to a concentration of said at least one element for inducing production by said cells of an amount of said compound of interest at least 2 times greater than an amount produced by said cells under identical culture conditions in a culture medium containing said at least one element at a minimum concentration for an optimal growth of said cells.

2. The method as claimed in claim 1, wherein the defensin is a defensing of a plant origin encoded by a gene of a Plant Defensin type 1 family having at least one of a three-dimensional structure comprising a CSαβ type motif, a gamma-core loop, and at least 30% amino acid identity with a defensin AhPDF1.1b of Arabidopsis halleri of sequence SEQ ID NO:2.

3. The method as claimed in claim 1, wherein said at least one element is zinc.

4. The method as claimed in claim 1, wherein said at least one element is present in said enriched culture medium at a concentration greater than or equal to the concentration of said at least one element for inducing the production by said cells of an amount of said compound of interest at least five times greater than the amount produced by said cells under identical culture conditions in said culture medium containing said at least one element at the minimum concentration for the optimal growth of said cells.

5. The method as claimed in claim 1, wherein said eukaryotic organism is chosen from a group consisting of yeasts, fungi, algae, microalgae, plants and animals.

6. The method as claimed in claim 1, wherein the cells of said eukaryotic organism are recombinant cells into which a nucleotide sequence coding for said defensin has been introduced.

7. The method as claimed in claim 1, further comprising a subsequent step of collecting the cells of said eukaryotic organism having accumulated said compound of interest.

8. The method as claimed in claim 1, wherein the compound of interest is a final compound in the sterol biosynthesis pathway.

9. The method as claimed in claim 1, wherein the compound of interest is an intermediate compound in the sterol biosynthesis pathway and said cells of said eukaryotic organism are recombinant cells modified to inhibit an enzyme activity involved in a biotransformation of said intermediate compound in the sterol biosynthesis pathway.

10. The method as claimed in claim 1, wherein the compound of interest is a compound derived from a compound of the sterol biosynthesis pathway, and said cells of said eukaryotic organism are recombinant cells modified to activate an enzyme activity involved in a biotransformation of said compound of the sterol biosynthesis pathway into said compound of interest.

11. A recombinant eukaryotic cell modified to express or overexpress a defensin and modified to inhibit an enzyme activity involved in a biotransformation of an intermediate compound in a sterol biosynthesis pathway in said recombinant eukaryotic cell.

12. The recombinant eukaryotic cell as claimed in claim 11, modified to activate an enzyme activity involved in the biotransformation of said intermediate compound into a compound of interest.

13. A method of using a recombinant yeast strain modified to express or overexpress a defensing for production of a compound of interest of a sterol biosynthesis pathway or derived from a compound of the sterol biosynthesis pathway in said recombinant yeast strain.

14. The method as claimed in claim 13, wherein cells of said recombinant yeast strain are subjected to a step of in vitro culture in an enriched culture medium configured to grow said cells and containing at least one element chosen from a group consisting of transition metals, lead and selenium, at a concentration greater than or equal to a concentration of said at least one element for inducing the production by said cells of an amount of said compound of interest at least 2 times greater than an amount produced by said cells under identical culture conditions in a culture medium containing said at least one element at a minimum concentration for an optimal growth of said cells.

15. The method as claimed in claim 13, wherein the defensin is a defensing of a plant origin encoded by a gene of a Plant Defensin type 1 family having at least one of a three-dimensional structure comprising a CSαβ type motif, a gamma-core loop, and at least 30% amino acid identity with a defensin AhPDF1.1b of Arabidopsis halleri of sequence SEQ ID NO:2.

16. The method as claimed in claim 1, comprising subsequent steps of collecting the cells of said eukaryotic organism having accumulated said compound of interest; and extracting said compound of interest.

17. The method as claimed in claim 10, wherein said cells of said eukaryotic organism are said recombinant cells modified to inhibit the enzyme activity involved in the biotransformation of an intermediate compound in the sterol biosynthesis pathway.

18. The method as claimed in claim 4, wherein said at least one element is present in said enriched culture medium at a concentration greater than or equal to the concentration of said at least one element for inducing the production by said cells of an amount of said compound of interest at least 10 times greater than the amount produced by said cells under identical culture conditions in said culture medium containing said at least one element at the minimum concentration for the optimal growth of said cells.


SEQUENCE LISTING (8 sequences)

SEQ ID NO: 1 - DNA, 243 bp, Arabidopsis halleri
atggctaagtttgcttccatcatcacccttatcttcgctgctcttgttctctttgctgcatttgaagcaccaacaactgtggaagcacagaggttgtgcgagaagccaagtgggacatggtcaggagtttgcggaaacaatggcgcgtgcagaaatcagtgcattagacttgagaaagcacgacatggatcttgcaactacgtcttcccagctcataagtgtatctgttacttcccatgttaa

SEQ ID NO: 2 - protein, 80 aa, Arabidopsis halleri
MAKFASIITLIFAALVLFAAFEAPTTVEAQRLCEKPSGTWSGVCGNNGACRNQCIRLEKARHGSCNYVFPAHKCICYFPC

SEQ ID NO: 3 - DNA, 237 bp, Arabidopsis halleri
atggcttcttcttacacccttatgctcttcctttgcctctccatcttccttatcgcttctactgagatgatggcggtggaagcaagaatttgcgagaggcgaagcaagacatggacagggttttgtggcaacactcgtggctgtgacagtcagtgtaagagttgggaacgtgcttcacacggggcttgtcacgctcagttcccgggtttcgcatgtttctgctacttcaattgctga

SEQ ID NO: 4 - protein, 78 aa, Arabidopsis halleri
MASSYTLMLFLCLSIFLIASTEMMAVEARICERRSKTWTGFCGNTRGCDSQCKSWERASHGACHAQFPGFACFCYFNC

SEQ ID NO: 5 - DNA, 682 bp, Triticum aestivum
ggcacgaggccacgtagacgcagcagcagcagcagcgagctctgtcggttctctatctagcatctccggtgtgaagcgagcaagcagagagagagagagatggcgtccactcgtcgcatggctgccgcgccggccgtcctccttctcctcctcctgctcgtcgccacagagatggggacgatgaagacggcggaggcccggacgtgcctgtcgcagagccacaagttcaagggcacctgcctcagcaacagcaactgcgccgccgtgtgccgcaccgagaacttccccgacggcgagtgcaacacgcacctcgtcgagcgcaagtgctactgcaagcggacctgctaagcaacaccagtccgcgcgacgtcctccgatggatgcttgctactgggctagagcatccatgccgtcccttagatctgttcgtccctactccgtctatttccgtttcctttacgttgctcctagggatgactgaaaataaagtagctacctgcaccctcggcattggctgttcgtctgcatgtatgctgtttaagtgttcgtggctttagtttgtgctgttgatgtaataacgatgccactgacaatttggcttctatgtcttgtgttggaactttgaatcagcaggtttgttggcttgtctgttaaaaaaaaaaaaaaaaaaaaaaaaaaaaaa

SEQ ID NO: 6 - protein, 82 aa, Triticum aestivum
MASTRRMAAAPAVLLLLLLLVATEMGTMKTAEARTCLSQSHKFKGTCLSNSNCAAVCRTENFPDGECNTHLVERKCYCKRTC

SEQ ID NO: 7 - DNA, 219 bp, Medicago sativa
atggagaagaaatcactagcaggcttatgcttcctcttcctcgttctctttgttgcacaagaaattgtggtgactgaagctaagacttgtgagaatttggctgataaatacaggggaccatgctttagtggttgtgatactcactgcactaccaaagagaatgcagttagcggcaggtgcagggatgactttcgttgttggtgtactaaaagatgttaa

SEQ ID NO: 8 - protein, 72 aa, Medicago sativa
MEKKSLAGLCFLFLVLFVAQEIVVTEAKTCENLADKYRGPCFSGCDTHCTTKENAVSGRCRDDFRCWCTKRC